(19) United States
(12) Patent Application Publication
(10) Pub. No.: US 2024/0282405 A1
(43) Pub. Date: Aug. 22, 2024

(54) ANALYSIS SYSTEM, LEARNED MODEL GENERATION DEVICE, DISCRIMINATION SYSTEM, ANALYSIS METHOD, LEARNED MODEL GENERATION METHOD, AND DISCRIMINATION METHOD

(71) Applicant: KONICA MINOLTA, INC., Chiyoda-ku, Tokyo (JP)

(72) Inventors: Ken OKAMOTO, Hachioji-shi, Tokyo (JP); Takatoshi SUEMATSU, Nerima-ku, Tokyo (JP); Wataru ONODA, Suginami-ku,Tokyo (JP); Atsuro TATSUMI, Sagamihara-shi, Kanagawa (JP); Yuichiro ASAI, Hino-shi, Tokyo (JP); Naoko FURUSAWA, Hino-shi, Tokyo (JP)

(73) Assignee: KONICA MINOLTA, INC., Chiyoda-ku, Tokyo (JP)

(21) Appl. No.: 18/567,367

(22) PCT Filed: Jun. 23, 2022
(86) PCT No.: PCT/JP2022/025204

§ 371 Date: Dec. 6, 2023

(30) Foreign Application Priority Data
Jun. 25, 2021 (JP) 2021-106124
Mar. 14, 2022 (WO) PCT/JP2022/011374

(51) Int. Cl. G16B 15/20; G01N 27/414; G16B 40/10

(52) U.S. Cl. CPC G16B 15/20; G01N 27/414; G16B 40/10

(57) ABSTRACT

An analysis system, a learned model generation device, a discrimination system, an analysis method, a learned model generation method, and a discrimination method can sensitively quantify at least one of an aggregation state or a surface state. An analysis system includes an acquisition unit for acquiring a signal change over time due to interaction between a capture target substance and a capture substance by an electrical detection method or optical detection method, and an analysis unit for analyzing at least one of an aggregation state and a surface state of the capture target substance from the signal change over time acquired by the acquisition unit.

### TECHNICAL FIELD

The present invention relates to an analysis system, a learned model generation device, a discrimination system, an analysis method, a learned model generation method, and a discrimination method.

### BACKGROUND

### (For Achieving SDGs)

Corporate activities for achieving SDGs (Sustainable Development Goals) are essential to maintain businesses continuously growing. Among the 17 goals of SDGs, the present invention mainly targets Goal 3: good health and well-being, and Goal 9: industries, innovation and infrastructure. The present invention addresses contributing to good health and well-being and building resilient infrastructure, to solve social issues.

### (SDGs3: Good Health and Well-being)

The present Invention addresses good health and well-being for the goal of the SDGs 3, and the preamble of the constitution of The World Health Organization defines that health is a state of complete physical, mental and social well-being and not merely the absence of diseases or infirmity. Therefore, it is required to contribute to the suppression/treatment of dementia and infectious diseases, and the development/quality control of pharmaceutical products.

### (SDGs 9: Industries, Innovation and Infrastructure)

With respect to Goal 9 of SDGs, in order to support economic growth and well-being, it is required to develop a sustainable and resilient infrastructure with high quality and reliability including an area/over border infrastructure. As resilient infrastructure, the present invention proposes a method that contributes to visualization of conditions, quality control, and evaluation of drug efficacy in the field from medical applications to products of precision industry.

### (DX)

On the other hand, it is important to make a new super-smart society “Society 5.0” happen in reality. Society 5.0 includes the philosophy of Industry 4.0 (the Fourth Industrial Revolution) proposed in Germany and is brought by Connected Industries in which a new use of AI and robots is recognized as means and various things and actions are connected. In order to do so, cyberspaces need to be effectively used so that non-visualized worlds can be visualized and next solutions can be obtained quickly. It is important that using machine learning as a method propels DX (Digital Transformation: an innovation by using digital technique) resulting in making it happen.

### FIELD OF THE INVENTION

In order to achieve the goals, SDGs 3 and 9, the present inventors have focused on nanomaterials. It is known that the aggregation state and a surface state in a nanomaterial affect the function and quality of the material. Nanomaterials include, for example, biomolecules (peptides, proteins, antibodies, and the like), organic compounds, and nanoparticles, and there are a wide variety of such materials. Nanomaterials have been used in a wide range of fields from the industrial field to the cosmetic/medical field, and have also become important key materials.

One of the proteins exemplified as the nanomaterial is amyloid β(Aβ). Aβ is one of the proteins involved in the progression of Alzheimer's dementia. For the measurement and evaluation of Aβ, management of the aggregation state and the surface state is also important. In addition, the control of the particle diameter of metal/inorganic nanoparticles for precision industrial products in a single nanometer (nm) order is important as well as the management of the aggregation state and the surface state is important since the particle diameter greatly affects the function.

Conventionally, as a method of measuring or evaluating the aggregation state or the surface state, a plurality of methods are known as described in Non-Patent Document 1. The Non-Patent Document 1 describes that there are a plurality of methods for detecting aggregates, for example, analysis by gel filtration chromatography, analysis by ThT fluorescence, analysis by a spectrophotometer, analysis by Dynamic Light Scattering (DLS), analysis by Differential Scanning Calorimetry (DSC), analysis by a microscope and the like. In addition, the Non-Patent Document 1 describes that there are problems in quantitativity and convenience in using these methods for quality control in a biopharmaceutical production site.

Furthermore, conventionally, as a method for measuring or evaluating these aggregation state and surface state, a technology described in Patent Document 1 has been proposed. Specifically, Patent Document 1 proposes an apparatus and a method for preparing a lipoprotein such as HDL, LDL, Lp (a), IDL, and VLDL from a biological sample using a differential charged particle mobility analysis method for diagnostic purposes. Furthermore, Patent Document 1 describes a method for analyzing the size distribution of the lipoprotein prepared by the above-described method by differential charged particle mobility.

As a method for detecting organic compounds such as proteins and nanomaterials such as nanoparticles, mass spectrometry, Size Exclusion Chromatography (SEC), simple evaluation methods, Western blotting, ELISAs (Enzyme-Linked Immuno Sorbent Assay), Surface Plasmon-field enhanced Fluorescence Spectroscopy (SPFS) and the like are known.

### RELATED ART

### Patent Literature

Patent Literature 1: JP 2010-529463 A

### Non-Patent Literature

Non-Patent Literature 1: Tatsuya Yoshino, “Evaluation of Aggregation States of Monoclonal Antibodies by Various Methods”, Doctoral Dissertation of Graduate School of Gunma University ([URL] https://gair.media.gunma-u.ac.jp/dspace/bitstream/10087/6652/1/Ph.D %20E-429.pdf [Search Date: Mar. 19, 2021]).

### SUMMARY

### Problems to be Solved

As described above, quantitative determination, simplicity, and the like remain problems in any of the methods described in Non-Patent Literature 1 in order to use them for quality control in a biopharmaceutical production site.

In addition, the technology described in Patent Literature 1 measures lipoproteins by particle mobility. Although a distribution size can be observed to some extent by using the technology, the accuracy is not sufficient.

In addition, the mass spectrometry has a disadvantage that an aggregation state is dissolved in a pretreatment/detection process, thereby a monomer and an aggregate cannot be distinguished from each other. The size exclusion chromatography, the dynamic light scattering method, and the simple evaluation method have a disadvantage that detection is impossible in a low concentration range. In addition, the dynamic light scattering method also has a disadvantage in that a detectability of a single nm order or less is low. The Western blotting method, the ELISA method, and the SPFS method require capture substances corresponding to a plurality of aggregation states, respectively, and have a disadvantage that preparing capture substances corresponding to all the aggregates is difficult. Note that there is a disadvantage that no means for detecting a nanomaterial such as a nanoparticle exists particularly on the order of a single nm or less with high sensitivity and no means for detecting a particle diameter and a surface state at the same time exists.

Therefore, although it is known that a size change (aggregation) of a biomarker influences on the health condition and the quality of an antibody pharmaceutical product, there is no means for quantitatively determining the aggregation distribution with high sensitivity. In addition, regarding nanomaterials, it is said that the function of nanomaterials is greatly changed by controlling the particle size and surface state, but there is no or limited means for detecting a size of single nm or less or a material to be detected with high sensitivity.

An object of the present invention is to provide an analysis system, a learned model generation device, a discrimination system, an analysis method, a learned model generation method, and a discrimination method that quantify at least one of an aggregation state or a surface state with high sensitivity.

### Solution to Problems

As a result of intensive studies to solve the above-mentioned problems, the present inventors have found that a plurality of pieces of aggregation information and surface information can be discriminated in one reaction field by acquiring a signal change over time due to an interaction between a capture target substance and a capture substance by an electrical or optical detection method, and have completed the present invention.

That is, the aforementioned problem according to the present invention is solved by the following means. 1. An analysis system comprising: an acquisition unit that acquires a signal change over time due to an interaction between a capture target substance and a capture substance by an electrical or optical detection method; and an analysis unit that analyzes at least one of an aggregation state and a surface state of the capture target substance from the signal change over time acquired by the acquisition unit.

2. A learned model generation device comprising: an acquisition unit for acquiring a signal change over time due to an interaction between a capture target substance and a capture substance by an electrical or optical detection method; an analysis unit for analyzing at least one of an aggregation state and a surface state of the capture target substance from the signal change over time acquired by the acquisition unit; and a machine learning unit for performing machine learning using a plurality of analysis results obtained by the analysis unit to generate a learned model.

3. A discrimination system including a discrimination unit configured to discriminate an acquired unknown signal change over time using the learned model generated by the learned model generation device according to 2, and output a discrimination result of at least one of an aggregation state and a surface state of a capture target substance having the unknown signal change over time.

4. An analysis method comprising: an acquisition step of acquiring a signal change over time due to interaction between a capture target substance and a capture substance by an electrical or optical detection method; and an analysis step of analyzing at least one of an aggregation state and a surface state of the capture target substance from the signal change over time acquired in the acquisition step.

5. A learned model generation method comprising: an acquisition step of acquiring a signal change over time due to an interaction between a capture target substance and a capture substance by an electrical or optical detection method; an analysis step of analyzing at least one of an aggregation state and a surface state of the capture target substance from the signal change over time acquired in the acquisition step; and a machine learning step of performing machine learning using a plurality of analysis results obtained in the analysis step to generate a learned model.

6. A discrimination method comprising a discrimination step of discriminating an acquired unknown signal change over time by using the learned model generated by the learned model generation method according to 5, and outputting a discrimination result of at least one of an aggregation state and a surface state of a capture target substance having the unknown signal change over time.

### Advantageous Effects of Invention

The present invention can provide an analysis system, a learned model generation device, a discrimination system, an analysis method, a learned model generation method, and a discrimination method that quantify at least one of an aggregation state or a surface state with high sensitivity.

### BRIEF DESCRIPTION OF DRAWINGS

FIG. 1 is a schematic view showing a configuration of an analysis system according to an embodiment of the present invention.

FIG. 2A is an explanatory diagram for explaining a detection example using an electrical detection signal.

FIG. 2B is an explanatory diagram for explaining a detection example by an SPFS signal.

FIG. 3A shows graphs. The left graph indicates a change in the threshold voltage Vth corresponding to a change in the binding rate over time at each concentration of the capture target obtained by the electrical detection signal. The left graph indicated that K_{d} can be calculated from the height Vthₘₐₓ (maximum binding rate) calculated from the left graph and the concentration of the capture target.

FIG. 3B shows that kₒₙ and k_{off} can be calculated. The left graph indicates a change in the threshold voltage Vth corresponding to a change in the binding rate of the capture target substance over time obtained from an electrical detection signal and the rate constants. The right graph indicates that kₒₙ and k_{off} can be calculated from the rate constants calculated from the left graph and the concentration of the capture target substance.

FIG. 4A is a graph showing an example of the kₒₙ, k_{off} becoming slower depending on the aggregation size when K_{d} is at a fixed time and of the change of aggregation sizes over time when the binding rate is fixed.

FIG. 4B is a table showing K_{d}, kₒₙ, k_{off} obtained from the results shown in FIG. 4A.

FIG. 5 is a conceptual diagram explaining the state of having a plurality of arrayed reaction fields (capture substances).

FIG. 6 is a schematic view showing an example of a device configuration for observing and evaluating an electrical detection signal over time.

FIG. 7 is a graph illustrating an example of the relationship between age and changes in biomarkers in Alzheimer's dementia.

FIG. 8 is a schematic view for explaining the configuration of an SPR device employing a continuous flow method.

FIG. 9 is a schematic view illustrating an example of a transistor-type biosensor.

FIG. 10 is a schematic view showing another example of the transistor-type biosensor.

FIG. 11 is a schematic view for explaining the arrangement of a learned model generation device according to an embodiment of the present invention.

FIG. 12 is a schematic view for explaining the arrangement of a discrimination system according to an embodiment of the present invention.

FIG. 13 is a flowchart illustrating an analysis method according to an embodiment of the present invention.

FIG. 14 is a flowchart illustrating a learned model generation method according to an embodiment of the present invention.

FIG. 15 is a flow chart illustrating a discrimination method according to an embodiment of the present invention.

FIG. 16 is a graph showing temporal changes in threshold value voltage Vth of a low aggregation sample (Aβ1-42 monomer) and a high aggregation sample (Aβ1-42 aggregate).

FIG. 17 is a graph which illustrates the changes over time in the threshold value voltage Vth of low aggregation samples (Aβ1-42 monomer) and high aggregation samples (Aβ1-42 aggregate) which have been prepared at various concentrations.

FIG. 18 is a graph illustrating the results of learning and classification prediction by a support vector machine (SVM) that is a machine learning model, using data obtained by dimensionally compressing data on the change over time of a threshold value voltage Vth by principal component analysis as an explanatory variable, and using whether a sample is a low aggregation sample or a high aggregation sample as an objective variable.

### DETAILED DESCRIPTION OF EMBODIMENTS

As described above, it is known that the aggregation state and the surface state of a nanomaterial affect the function and quality of the material.

The present invention provides a system for quantifying an aggregation distribution and a surface state of a nanomaterial with high sensitivity. Specifically, by acquiring a signal change over time due to the interaction between the capture target substance and the capture substance by an electrical or optical detection method, a plurality of pieces of aggregation information and surface information can be discriminated in one reaction field.

Hereinafter, the present invention, constituent elements thereof, and modes and aspects for carrying out the present invention will be described in detail with reference to the drawings as appropriate. Hereinafter, embodiments for carrying out the present invention will be described in detail, but the present invention is not limited thereto.

### <Analysis System>

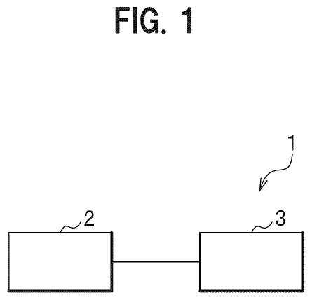

First, an analysis system according to an embodiment of the present invention will be briefly described. FIG. 1 schematically illustrates a configuration of an analysis system 1 according to an embodiment of the invention.

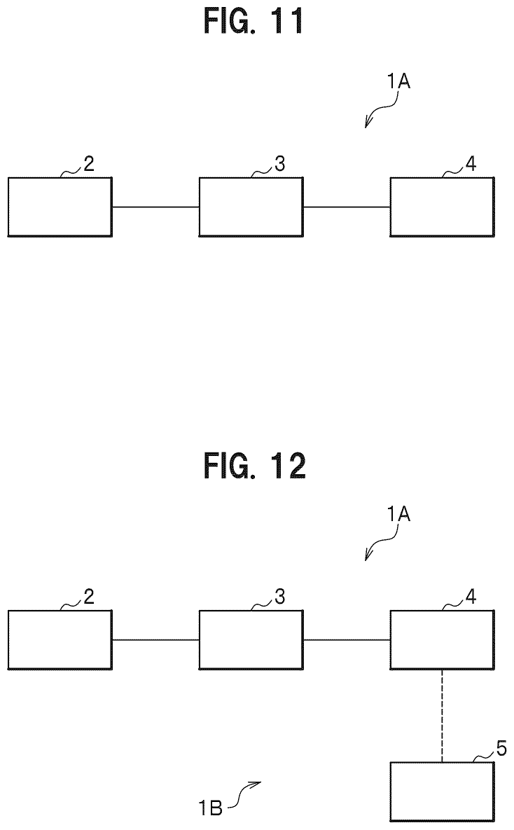

As illustrated in FIG. 1, the analysis system 1 includes an acquisition unit 2 and an analysis unit 3. The analysis system 1 is used in a learned model generation device 1A (FIG. 11) and a discrimination system 1B (FIG. 12) described later.

### (Acquisition Unit)

The acquisition unit 2 acquires a signal change over time due to the interaction between the capture target substance and the capture substance by an electrical or optical detection method. In the present specification, “over time” refers to the order of time that elapses, and the order of time may be continuous or intermittent. As such an acquisition unit 2, for example, a Surface Plasmon Resonance (SPR), Field Effect Transistor (FET) or the like are included. Note that the SPR device and the transistor-type biosensor will be described later. Other than these, examples of such acquisition unit 2 include, for example, a competition method utilizing a fluorescence signal or an absorption signal.

### (Aspect of Acquisition Unit)

Here, one aspect of the acquisition unit 2 will be described.

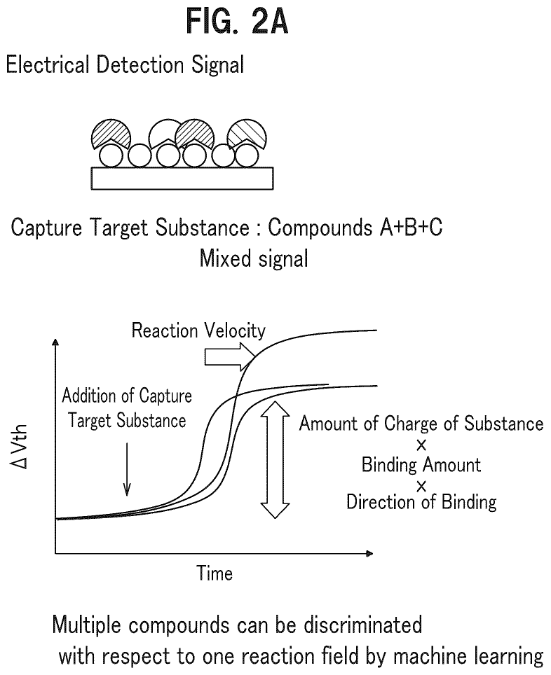

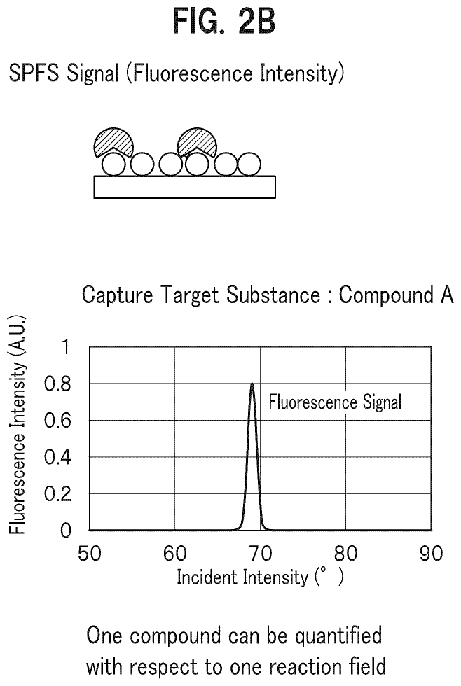

FIG. 2A is an explanatory diagram for explaining an example of detection using an electrical detection signal. FIG. 2B is an explanatory diagram for explaining an example of detection using an SPFS signal.

As shown in FIG. 2A, in the electrical detection signal obtained by the SPR device or the transistor-type biosensor, a signal change over time after the addition of the capture target substance can be detected. The change over time may be in the order of time as in the above-described change over time, and may be continuous detection and a detection signal resulting therefrom, or may be intermittent detection and a detection signal resulting therefrom. The electric detection signal can be discriminated by separating and quantifying a mixed signal of a plurality of capture target substances (compounds A+B+C). That is, a mixed signal of a plurality of compounds is obtained with respect to one reaction field (one signal) as the electrical detection signal, and thus information on the plurality of compounds can be obtained. As shown in FIG. 2B, only one compound can be quantified with respect to one reaction field (one signal) by the SPFS signal (fluorescence intensity). Therefore, it can be said that the electric detection signal described above is more suitable than the SPFS signal when a mixed signal of a plurality of compounds is discriminated by machine learning as described later.

### (Electrical Detection Signal and Capture Substance)

The electrical detection signal will be further described.

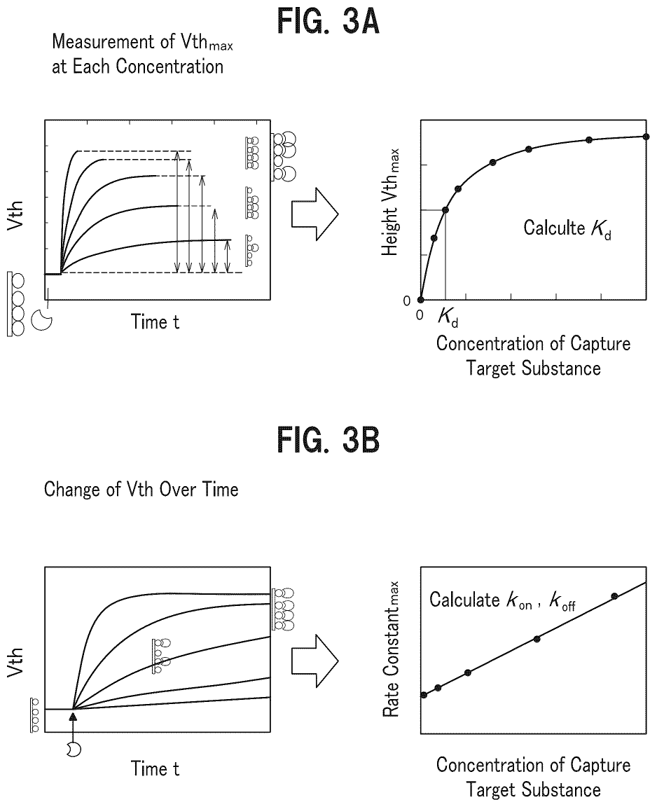

Regarding the electrical detection signal, a mixed signal of a plurality of compounds can be obtained over time with respect to one reaction field (one signal). Therefore, as shown in FIG. 3A and FIG. 3B, K_{d} (dissociation constant (unit: M)), Kₒₙ (binding rate constant (unit: M⁻¹s⁻¹), K_{off} (dissociation rate constant (unit: s⁻¹) can be calculated. FIG. 3A shows graphs. The left graph indicates a change in the threshold value voltage Vth corresponding to a change in the binding rate over time at each concentration of the capture target substance obtained by the electrical detection signal. The right graph indicates that a height Vthₘₐₓ (maximum binding rate) calculated from the left graph, and that K_{d} can be calculated from the concentration of capture target substance. Further, FIG. 3B shows graphs in which the left graph indicates a change in the threshold value voltage Vth corresponding to a change in the binding rate over time of the capture target substance obtained by the electrical detection signal, and the right graph indicates that kₒₙ and k_{off} can be calculated from the rate constant calculated from the left graph and the concentration of the capture target substance. In the right graph of FIG. 3B, the slope is kₒₙ and the intercept is k_{off}.

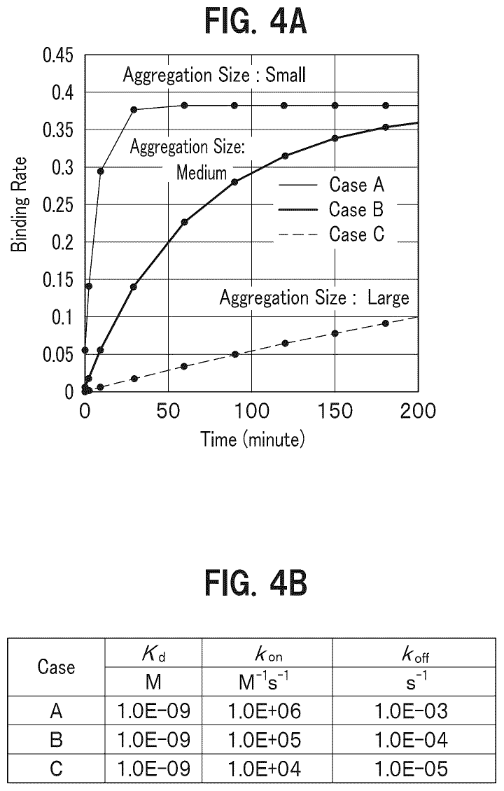

The physical parameter and the binding amount related to the interaction between the capture substance and the capture target substance change depending on the aggregation size. Thus, the electrical detection signal (height, velocity) changes. FIG. 4A is a graph showing an example that the kₒₙ, k_{off} is delayed depending on small, medium, and large aggregation sizes (cases A, B, and C) when the dissociation constant K_{d} is constant and changes of the aggregation sizes over time when the binding rate is constant. The results shown in FIG. 4B are tables showing K_{d}, kₒₙ, k_{off} obtained from the results shown in FIG. 4A. As shown in the graph of FIG. 4A and the table of FIG. 4B, in a case where the size of the aggregate increases, at least a change of kₒₙ (slow) and k_{off} (slow) occurs, and a signal change occurs. In addition, Vthₘₐₓ also changes.

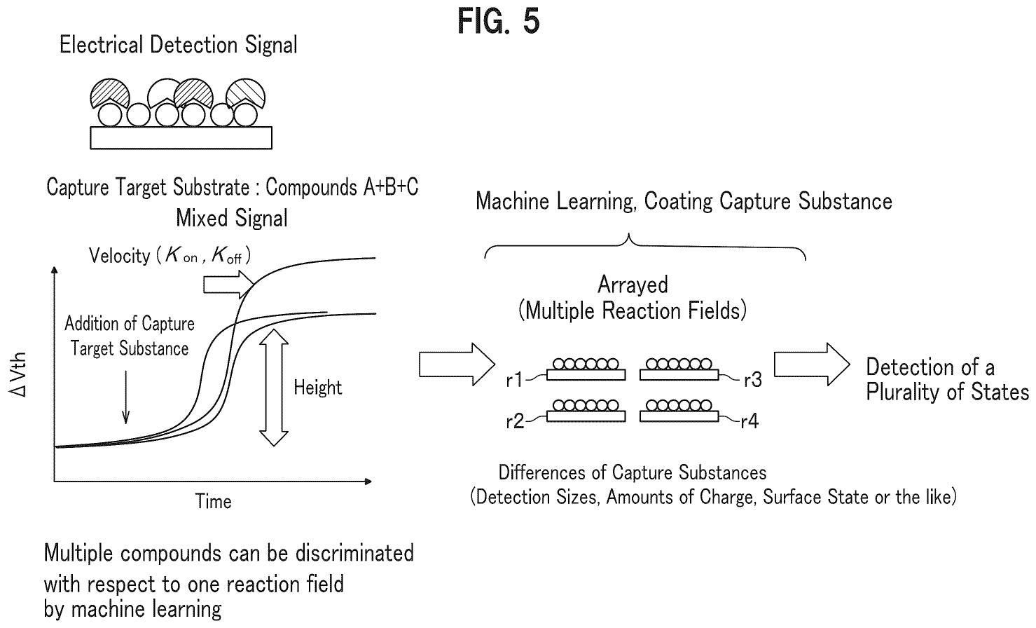

Further, in the reaction between one capture substance and the aggregate mixture, a signal (mixed signal) in which electric detection signals corresponding to the respective aggregates are mixed can be obtained. From the mixed signal, components of height (binding rate (amount of change in Vth)) and velocity (binding rate constant (kₒₙ), dissociation rate constant (k_{off})) can be separated. Then, the component prediction of the aggregation size can be performed by machine learning from the separated components (signal change). As an example, as shown in FIG. 5, in the arrayed first reaction field, as described with reference to FIG. 4A of the drawing, the capture substance r1 for recognizing the aggregation size is set. As shown in FIG. 5, in the arrayed second, third, and fourth reaction fields, the types of the capture substances (different range zones of sizes of aggregates that interact with the capture substances) may be changed to obtain results (detection of a plurality of states), such as the capture substances r2 and r3 for sensing different range zones of sizes, the capture substances r4 for detecting a change in the amount of charge, and the like, and these may be used in combination. Then, machine learning is performed using the result. There are factors such as the non-equilibrium and direction of charge at the time of aggregation, and there is a possibility that a complex equilibrium reaction occurs at the time of mixing the capture target substance and results in a complex signal, but a change point thereof is identified and discriminated by machine learning. By doing so, it is possible to obtain information on the aggregation distribution and the surface state more widely, finely, and accurately. FIG. 5 is a conceptual diagram for explaining a state in which a plurality of arrayed reaction fields (capture substances) are provided.

As the acquisition unit 2, a measurement apparatus using an electric signal is preferable from the viewpoint that a large amount of signal information with respect to aggregates can be acquired and binding rate and binding rate information for each size can be acquired. As the acquisition means 2, for example, as described above, a transistor-type biosensor using an FET or the like, an SPR device, or the like is preferable. Both of these can follow changes in a signal over time. It is also effective to adopt, as the acquisition unit 2, a method of taking temperature dependence, intentionally causing a velocity change, and increasing the amount of information.

### (Method for Evaluating Electric Detection Signal)

An example of a method for evaluating an electrical detection signal will be described. For example, an electrical detection unit (transistor-type biosensor) using an FET is used, a capture substance is formed on a gate part, and a site where an interaction between the capture target substance and the capture substance occurs at the time of adding the capture target substance is formed. By doing so, the semiconductor parameter analyzer can follow a change in ΔVth (difference between the initial value and the threshold value voltage Vth) over time along with the charge change of the gate part. Therefore, the semiconductor parameter analyzer can observe and evaluate a change in the interaction between the capture target substance and the capture substance over time. FIG. 6 is a schematic view showing an example of a device configuration for observing and evaluating an electrical detection signal over time. In the example shown in FIG. 6, the gate part is used as an extension gate, and the capture substance is formed on the Au electrode. In the example shown in FIG. 6, an Au electrode and a reference electrode are disposed in a container containing a predetermined measurement solution and are brought into contact with the measurement solution. When the capture target substance is added to the measurement solution, as described above, the semiconductor parameter analyzer can follow the change in ΔVth over time. Measurement with the semiconductor parameter analyzer can be performed with a drain-source voltage (voltage Vds) fixed and a gate-source voltage (voltage Vgs) variable.

### (Region of Capture Target Substance)

Examples of the region to which the capture target substance belongs include a region in which the aggregation state and the surface state affect the function and quality of a material, the biological permeability, and the degree of progress of disease in a wide range from medical applications to industrial applications. In terms of medical applications, Aβ peptides (proteins), antibodies, beads with antibodies, fluorescent nanoparticles (PID (Phosphor Integrated Dot) particles), liposomes with polyethylene glycol (PEG), and the like can be the capture target substances. In addition, in industrial application, metal nanoparticles, carbon nanotubes, magnetic fluid, nanosilica (sealing filler or the like), crystalline zirconia, and the like can be substances to be captured. Among these, Aβ peptides (proteins), antibodies, metallic nanoparticles, carbon nanotubes, magnetic fluids, nanosilica (sealing filler and the like), crystalline zirconia and the like have a size of a single nm order to a 10 nm order, and can be suitable capture target substances in the present embodiment.

### (Contribution to Alzheimer's Type Dementia)

An example of a substance to be captured that is regarded as promising is Aβ. It has been reported that aggregation of Aβ proceeds, accumulation of aggregates occurs in the brain, and then mild cognitive impairment and dementia occur. In the related art, in many cases, Positron Emission Tomography (PET) is used to detect Aβ, or a cerebrospinal fluid is collected and examined. However, PET imposes a large burden on a patient in terms of high costs, and an examination by collection of cerebral spinal fluid imposes a large burden on a patient in terms of invasiveness due to lumbar puncture in collection of cerebral spinal fluid. In addition, since the component (Aβ) flowing into blood is decomposed (it is suggested that an Aβ oligomer is soluble and is dissolved in blood), it is difficult to determine only by quantifying the specific component. Currently, immunoprecipitation-mass spectrometry (IP-MS), mild cognitive impairment (MCI) screening test, analysis by Simoa™ (single-molecule array), and the like, but they have not reached a level at which the degree of progress of dementia can be determined by Aβ in blood or the like.

With regard to Alzheimer's type dementia, the capture target substance in PET is Aβ aggregates (fibrils/fibers). The capture target in IP-MS is the ratio between Aβ1-42 and APP699-711 or Aβ1-40 in blood. The capture target substances in the MCI screening test are three proteins (C3, ApoA1, and TTR) involved in Aβ clearance in blood. The capture target substance in the analysis by Simoa™ is p-tau in blood. The detection concentration of each capture target substance in blood is as described in Table 1. Incidentally, “-” in Table 1 indicates that there is no matter to be noted about the detection concentration.

TABLE 1

| Target | Change in | | Detection | | |
|---|---|---|---|---|---|
| substance | AD | Patient | concentration | | Source of reference |
| Aβ aggregate | Increase | AD patient | — | | Accumulation in brains |
| Aβ1-42 | Increase | Early stage | 2.9-5.6 pM | | Alzheimers Res Ther |
| | | patient | (in blood) | | |
| | Decrease | AD Patient | <110 pM | | The Japanese Research Group |
| | | | (cerebrospinal | | on Senile Dementia p39-45, |
| | | | fluid) | | Vol21 No. 4 2017 |
| p-tau | Increase | AD patient | fM order | | AMED HP (SIMOA ™) |
| C3 | Decrease | Normal | 7.2 | nM | ELISA Kit |
| ApoA1 | | Normal | 29-64 | nM | ELISA Kit |
| TTR | | Normal | 14-21 | nM | ELISA Kit |

Both analyses by IP-MS and Simoa™ have a large burden on patients in terms of high costs, similarly to PET. In addition, as described above, the IP-MS and the MCI screening test have not reached a level at which the degree of progress of dementia can be determined by Aβ in blood or the like (the accuracy is not sufficient).

Therefore, if the distribution of Aβ aggregation in cerebrospinal fluid and blood can be visualized with high sensitivity, it is conceivable that it can be used for the determination of the degree of progression of dementia and the evaluation of drug efficacy. The analysis system according to the present embodiment acquires a time-dependent signal change due to the interaction between the capture target substance and the capture substance by an electrical or optical detection method. Since an SPR device, a transistor-type biosensor, or the like is used as the electrical or optical detection method as described above, capture target substances in an extremely low concentration range (for example, 10 fM to 100 μM) can be detected (aggregation distribution can be detected with high sensitivity). Accordingly, the analysis system according to the present embodiment can perform evaluations without concentrating Aβ in blood by using an electrical or optical detection method.

Several monomers of the Aβ protein aggregate to form oligomers, further aggregate to form prefibrils, and further aggregate to form Aβ fibrils. Congo red specifically binds to and stains Aβ fibrils. Therefore, also in the detection method using electricity or optics in the present embodiment, by utilizing this, the aggregation state of Aβ can be detected even in an extremely low concentration range. Furthermore, from this, if a low-molecular-weight compound having selectivity for an Aβ monomer or an Aβ oligomer is developed and searched, an Aβ monomer or an Aβ oligomer can also be detected in an extremely low concentration range. By doing so, it is possible to further improve the determination accuracy by detecting, verifying, and analyzing these in combination. Furthermore, it is also possible to further improve the determination accuracy by determining the accumulation of Aβ and detecting, verifying, and analyzing the accumulation of Aβ in combination with other related biomarkers (C3, ApoA1, TTR, and the like). For these reasons, the analysis system 1, the learned model generation device 1A, and the discrimination system 1B according to the present embodiment can contribute to examination and treatment of Alzheimer's dementia. At present, there is only a therapeutic drug that delays the progress, and it is important to perform early diagnosis/treatment in the treatment of dementia. In addition, currently, all of the capture target substances in the clinical trial are Aβ.

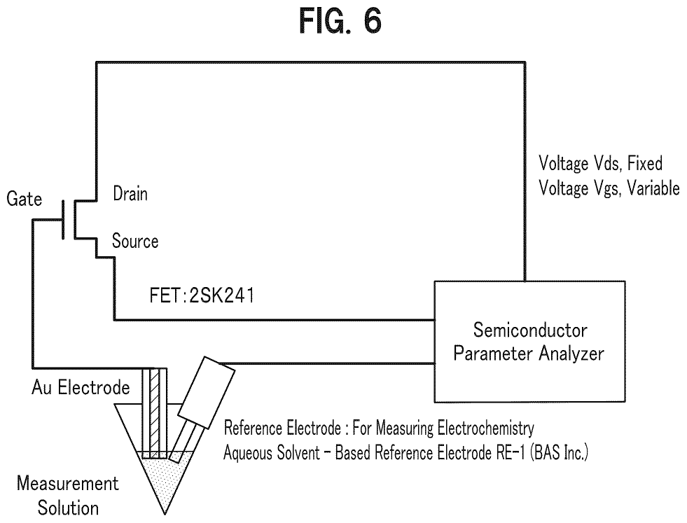

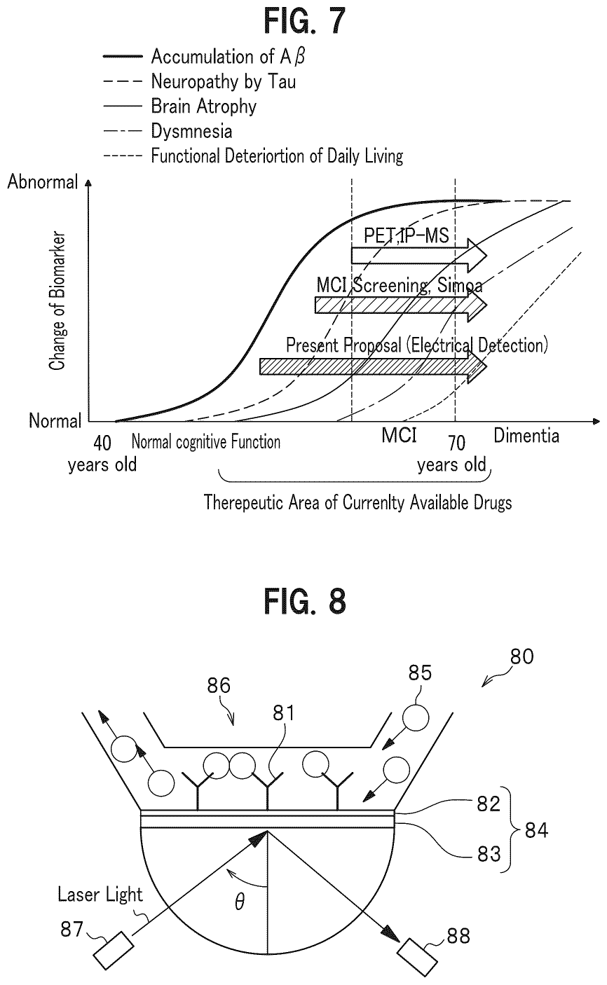

Here, FIG. 7 is a graph illustrating an example of the relationship between age and changes in biomarkers in Alzheimer's dementia. As shown in FIG. 7, the present proposals (the analysis system 1, the learned model generation device 1A, and the discrimination system 1B according to the present embodiment) can detect, analyze, and discriminate a low-concentration capture target substance. Therefore, the present proposal can be used in a pre-stage of causing brain atrophy or memory impairment and in MCI. For example, the present proposal can be used for early diagnosis and early intervention (determination of the degree of progression and evaluation of drug efficacy) from an even earlier stage than the analyses by PET, IP-MS, MCI screening test, and Simoa™. In addition, currently, development of a therapeutic agent using Aβ as a capture target substance for the purpose of suppressing early stage symptom deterioration is in progress, but the present proposal can also be used for this.

### (Aggregation Control/Real-Time Monitoring of Nanomaterial)

Nanomaterials are utilized in a variety of industrial/medical products. It is important to control the aggregation state of the liquid itself in terms of securing the quality of a product and visualizing a reaction mechanism. Therefore, the particle size distribution is measured by a method centering on a dynamic light scattering method. Problems with the dynamic light scattering method and other methods are that detection of a size equal to or smaller than a single nanometer order size is difficult, and evaluation can be performed only in a high concentration range zone where the concentration of a substance to be captured is mM to M. In addition, since the surface state is measured by a zeta potential and the particle diameter is measured by another means, there is a problem in simplicity of evaluation.

The present proposal is characterized in that the aggregation state and distribution can be evaluated even in a nanomaterial having a size of a single nm order or less and a low concentration zone in which the concentration of the capturing target substance is less than mM. In addition, since the detection signal due to the interaction between the capture target substance and the capture substance is acquired, the surface state can also be observed. For medical applications, quality control is important, including surface condition and particle size distribution. Therefore, the field of using nanomaterials is considered to be one of the promising fields for utilizing this proposal. It is desirable to detect not only the particle size but also the surface state and particle size distribution including crystallinity, molecular state, and stealth property (surface state including a modifying group) of nanoparticles for a drug delivery system, wound treatment, or the like. In addition, it is desirable to detect the surface state and the particle size distribution in the same manner for quality control of resin fillers for precision industrial products, and for metal catalysts and nano zeolites whose functions (fluidity, adsorption power, and catalytic properties) vary depending on the particle size and which require a small nano size for high functionality. Note that when the capture substance is a nanomaterial, as design principles for capturing this, 1 modified group) interaction with a modifying group, 2) size capture space (by size), and 3) interaction with a metal can be exemplified.

As an example of a case where the capture substance is a nanomaterial, a high-temperature lead solder substitute material which is a target of RohS regulation, more specifically, a die bonding material is exemplified. As a material for such a die bonding material, silver nanoparticles are a strong candidate. For a die bonding material using silver nanoparticles as a material, lowering of a firing temperature and improvement of bonding characteristics are required. As the particle size of the silver nanoparticles becomes smaller, the surface energy per area increases, and the melting point decrease. Therefore, a lowering of the firing temperature is achieved. In order to achieve these, a die bonding material using silver nanoparticles as a material is required to have a small particle size (2 nm or less) and a particle size distribution width (0.5 mm or less). However, the current evaluation methods do not sufficiently respond to the evaluation of the formation of particles. With the below-described analysis unit 3, the particle size distribution can be observed, and information on the surface state, crystallinity, and modifying chain can be acquired from the signal information. Further, the analysis unit 3, which will be described later, can determine the features of the signal height, velocity, and signal behavior for each corresponding content, and acquire information in which each content is distinguished. Therefore, in the present proposal, a signal change over time due to the interaction between the capture target substance and the capture substance is acquired, analyzed, or determined by an electrical or optical detection method capable of detecting a low concentration zone, and therefore, the present proposal is suitable for evaluation in such a region.

### (Contribution to Quality Inspection of Antibody Pharmaceutical Product)

A high concentration (50 to 100 mg/mL) liquid can also be evaluated by DLS, SEC, or the like, but it is known that there are problems in quantitative determination and simplicity. However, in the present proposal, since a signal change over time due to the interaction between the capture target substance and the capture substance is acquired, analyzed, or discriminated by an electrical or optical detection method capable of detecting a low concentration zone, an evaluation method having both quantitativeness and simplicity can be provided.

### (An Example of an Acquisition Unit: Surface Plasmon Resonance (SPR) Apparatus)

An example of the acquisition unit 2 is an SPR device as described above. As the SPR apparatus, a conventionally known general one, for example, a continuous flow type one can be used. FIG. 8 is a schematic view illustrating a configuration of the continuous flow SPR apparatus 80. As shown in FIG. 8, the SPR device 80 includes a sensor chip 84 having a metal thin film 82 (for example, a gold thin film) on which a capture substance 81 is immobilized and a glass substrate 83 provided in contact with the metal thin film 82. The SPR device 80 has a liquid feeding system 86 for bringing a capture target substance 85 (analyte) into contact with and binding to the capturing substance 81 of the sensor chip 84. The SPR device 80 has a light source 87 that irradiates the surface of the metal thin film 82 opposite to the surface on which the capture substance 81 is immobilized with laser light at a predetermined angle. The SPR device 80 includes an optical detector 88 that receives and detects the laser light applied from the light source 87 and reflected off the opposite surface.

### (Metal Thin Film)

As the metal thin film 82, the same metal as the metal thin film constituting a sensor chip used in a general SPR device can be used. That is, the metal thin film 82 is preferably formed of at least one metal selected from the group consisting of gold, silver, aluminum, copper, and platinum, and more preferably formed of gold among these. These metals may be in the form of an alloy thereof, or may be a laminate of metals.

The metal thin film 82 is preferably formed on a main surface of a dielectric member (not illustrated). As a method for forming the metal thin film 82 on the main surface of the dielectric member, a commonly used method can be used. As such a method, for example, the metal thin film 82 can be formed on the main surface of the dielectric member by a vacuum film-forming method such as an electron beam heating vacuum deposition method, a resistance heating vacuum deposition method, a magnetron sputtering method, a plasma-assisted sputtering method, an ion-assisted deposition method, or an ion plating method. The dielectric member can be formed of any material generally used for the sensor chip 84 used in the SPR device 80, for example, SiO₂, ZrO₂, SiₓN_{y}.

The thickness of the metal thin film 82 is preferably 5 to 500 nm for gold, 5 to 500 nm for silver, 5 to 500 nm for aluminum, 5 to 500 nm for copper, 5 to 500 nm for platinum, or 5 to 500 nm for alloys or laminates thereof. In terms of the electric field enhancement effect, 20 to 70 nm for gold, 20 to 70 nm for silver, 10 to 50 nm for aluminum, 20 to 70 nm for copper, 20 to 70 nm for platinum, and 10 to 70 nm for alloys or laminates of these metals are more preferable. When the thickness of the metal thin film 82 is within the above range, surface plasmon is easily generated, which is preferable.

### (Sensor Part)

The sensor chip 84 has a sensor part (not shown). The sensor part is provided in a partial region on the metal thin film 82 of the sensor chip 84, and the capture substance 81 is immobilized to this region. In this case, a plurality of sensor portions may be provided, and different capture substances 81 may be immobilized on the respective sensor parts.

The capture substance 81 is a substance that specifically captures the capture target substance 85. Examples of the capture substances 81 include antibodies to antigens such as Aβ protein; enzyme for substrate-coenzyme; receptors for hormones; protein A against antibodies; Protein G; avidins to biotin; calmodulin to calcium and lectin for sugars. When the capture target substance 85 is a nucleic acid, a nucleic acid having a sequence that specifically binds to the nucleic acid can be used as the capture substance 81. Furthermore, when the capture target substance 85 is a nanomaterial, a metal ion chelating agent, a crown ether, an ionophore group, or the like can be used as the capture substance 81.

Examples of the capture target substance 85 include, for example, proteins, lipids, sugars, nucleic acids, and other various substances, and in the case of the present embodiment, specific examples thereof include Aβ protein and nanomaterials.

As a method for immobilizing the capture substance 81 on the metal thin film 82, a commonly used method can be used. As such a method, for example, a modifying group that generates a specific bond is introduced into the surface of the metal thin film 82, a reactive group corresponding to this modifying group is introduced into the capture substance 81, and these modifying group and reactive group are bonded, thereby enabling the capture substance 81 to be immobilized on the metal thin film 82.

To be specific, for example, the surfaces of the metallic thin films 82 are treated with silane coupling agents having amino groups at the terminals to be modified with the amino groups, then treated with NHS (N-hydroxysuccinimide)-PEG4-biotin to bind biotin to the amino groups, and after avidin is reacted with the biotin, the biotinylated capture substances 81 (for example, antibodies) are reacted. By doing so, the capture substance 81 can be immobilized on the metal thin film 82. Furthermore, to be specific, for example, the surfaces of the metal thin films 82 are treated with silane coupling agents having carboxyl groups at their terminals to modify them with carboxyl groups, and are then treated with EDC (1-Ethyl-3-[3-dimethylaminopropyl] carbodiimide hydrochloride) and NHS to activate and esterify the carboxyl groups, and are then reacted with capture substances 81 (e.g., antibodies) having amino groups. By doing so, the capture substance 81 can also be immobilized on the metal thin film 82.

Optionally, a self-assembled monolayer (SAM) may be formed on the surface of the metal thin film 82, and the capture substance 81 may be immobilized on the metal thin film 82. The SAM has a role as a base when the capture substance 81 is immobilized on the metal thin film 82.

As the monomer included in the SAM, for example, carboxyalkanethiol having a carbon number of about 4 to 20 (e.g., available from Dojindo Laboratories, Sigma-Aldrich Japan Corporation), particularly preferably 10-carboxy-1-decanethiol, is used. A carboxyalkanethiol having 4 to 20 carbon atoms is preferable because an SAM formed using the carboxyalkanethiol has little optical influence, that is, the SAM has properties such as high transparency, a low refractive index, and a thin film thickness.

The method of forming the SAM is not particularly limited, and a well-known method of the related art such as an immersion method, an ink jet method, a dispenser, an applicator, a nozzle jet, or direct dropping (a pipette or a quantitative weighing machine) can be used. Specific examples thereof include a method of immersing the metal thin film 82 in an ethanol solution containing 10-carboxy-1-decanethiol (manufactured by Dojindo Molecular Technologies, Inc). A thiol group of 10-carboxy-1-decanethiol is bonded to metal and immobilized, and self-assembles on the surface of the metal thin film 82 to form a SAM. The method for immobilizing the capture substance 81 on the formed SAM is not particularly limited, and a method known in the related art can be used, and for example, the method of performing treatment with EDC and NHS can be used.

The solvent in which the SAM is dissolved or dispersed is not particularly limited, and the following solvents can be used. Such solvents include, for example, halogen-based solvent such as chloroform, carbon tetrachloride, dichloromethane, 1,2-dichloroethane, dichlorobenzene and dichlorohexanone, ketone-based solvent such as acetone, methyl ethyl ketone, diethyl ketone, methyl isobutyl ketone, N-propyl methyl ketone and cyclohexanone, aromatic solvent such as benzene, toluene, xylene, mesitylene and cyclohexylbenzene, aliphatic solvent cyclohexane, decalin and dodecane, ethereal solvent such as ethyl acetate, N-propyl acetate, N-butyl acetate, methyl propionate, ethyl propionate, γ-butyrolactone and diethyl carbonate, amide-based solvent such as tetrahydrofuran, ethereal solvent such as dioxane, dimethylformamide and dimethylacetamide, alcohol-based solvent such as methanol, ethanol, 1-butanol and ethylene glycol, nitrile-based solvent such acetonitrile and propionitrile, dimethyl sulfoxide, water, various buffers, or a mixed solvent thereof.

The boiling point of these solvents is preferably a boiling point lower than the temperature of the drying treatment from the viewpoint of rapidly drying the solvent, and specifically, it is preferably within a range of 60 to 200° C., more preferably within a range of 80 to 180° C.

The above-mentioned solvents may be used in combination in order to adjust the viscosity and the surface tension according to the method for forming the SAM.

The SAM solution (a solvent containing a single molecule for forming a SAM) can contain a surfactant according to the purpose of controlling an application range or the purpose of suppressing liquid flow (e.g., liquid flow that causes a phenomenon called coffee ring) due to a surface tension gradient after application. Examples of the surfactant include an anionic or nonionic surfactant from the viewpoint of the influence of moisture contained in the solvent, leveling properties, wettability to the substrate (metal thin film 82), and the like. Specific examples thereof include fluorine-containing surfactants and surfactants described in International Patent Publication No. 08/146681, Japanese Unexamined Patent Publication No. H 2-41308.

The solvent used for SAM may be a solution in which SAM is uniformly dissolved in a solvent or a dispersion liquid in which a material is dispersed in a solvent as a solid content. As the dispersion method, dispersion can be performed by a dispersion method such as ultrasonic wave, high shear force dispersion or media dispersion.

The viscosity of the SAM-solution can be appropriately selected depending on the solubility or dispersibility, and specific examples thereof can be selected within the range of 0.3 to 100 mPa s.

In the present embodiment, after the SAM solution is formed on the metal thin film 82, a drying step of removing the above-described solvent can be provided. The temperature of the drying step is not particularly limited, but it is preferable that the drying treatment is performed at a temperature at which the base material such as the metal thin film 82 is not damaged. Although the drying temperature varies depending on the composition of the SAM solution and the like, it cannot be said unconditionally, but for example, the drying temperature can be set to a temperature of 80° C. or higher, and it is considered that the drying temperature can be set to a temperature up to about 200° C. as the upper limit. The drying time is preferably set to an appropriate time (for example, at 80° C. for 30 minute) depending on the material such as the solvent used. Under such conditions, the drying can be rapidly performed.

It is also known that carbene ligands bind strongly to gold (Renee W. Y. Man et. al., J. Am. Chem. Soc. 2018, 140, 1576-1579). Therefore, in a case where a carbene ligand is used, the binding density of the capture substance 81 (for example, an antibody) to the metal thin film 82 (preferably a gold thin film) is improved due to the binding stability, and thus, an increase in sensitivity is expected. In a case where the carbene ligand is bonded to the metal thin film 82 and the capture substance 81 (for example, a labeled antibody) is bonded thereto in the same manner as described above, a configuration of the metal thin film 82—the carbene ligand-capture substance 81 can be obtained. In addition, regarding the coordination site of the carbene ligand, the bond between the bidentate ligand and the metal thin film 82 and the bond between the tridentate ligand and the metal thin film 82 are stronger than the bond between the monodentate ligand and the metal thin film 82, thereby the stability is enhanced. A bidentate ligand, a tridentate ligand, or a higher ligand is more preferable than a monodentate ligand in terms of stability and sensitivity enhancement.

In addition to carbene, silylene which is a silicon derivative, germylene which is a germanium analog, and the like can be used. Further, there is nitrene as a chemical species having two electrons less than the coordination saturation, and the nitrene can also be used in the present embodiment. Note that the following formulae (1) and (2) illustrate examples of the carbene ligand. In Formulas (1) and (2), R′ represents the capture substance 81.

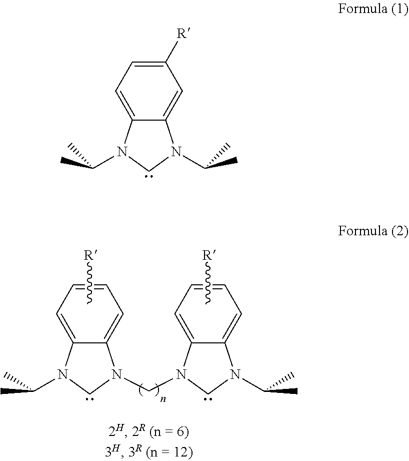

In the carbene ligand represented by Formula (1), (2), the element positioned at X may be other than N as in Formula (3), (4). Examples of the element located at X include, but are not limited to, di-coordinated carbon. Even with such a compound, an action similar to that of the carbene ligand can be expected. Note that R in the formulae (3) and (4) represents hydrogen, an alkyl group or an aryl group. M represents a metal. L_{X} represents a ligand, and X in L_{X} represents the number of the ligand.

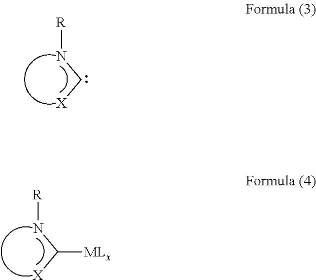

In the carbene ligands represented by the formulae (1) to (4), the cyclic structure moiety may be a 5-membered ring or a 6-membered ring. Examples of 5-membered rings include, but not limited to, azole, imidazole, pyrrole, thiophene, furan, pyrazole, oxazole, isoxazole, thiazole, triazole, and Pentazole. Examples of 6-membered rings include, but not limited to, pyridine, pyrimidine, pyridazine, triazine, tetrazine, pentazine and hexazin. All of the above-described carbene ligands and the like can be bonded to the surface of the metal thin film 82 (preferably the surface of the gold thin film).

The shape and area of the region where the capture substance 81 is immobilized on the metal thin film 82, i.e., the sensor part, are not particularly limited, but incident light (parallel light) is applied to the entire chip surface through a prism, and the reflected light is detected by a photodiode (PD), a charge-coupled device (CCD) detector, or the like. Only the interacted portion is observed as a brightly changed signal or image. In particular, in order to improve the S/N ratio at the time of measurement when excited by the sensor part, the shape of the sensor part is preferably the same as the shape of the region irradiated with the excitation light.

### (Labeling Agent)

The labeling agent is a complex containing a substance that specifically binds to the capture target substance 85 and a substance whose reflectance is changed upon irradiation with predetermined excitation light or a fluorescent body capable of emitting fluorescence upon irradiation with predetermined excitation light. For example, in a case where the capture target substance 85 is an antigen (for example, Aβ protein), a complex of a substance having a reflectance variation and an antibody specifically binding to the antigen, or a complex (labeled secondary antibody) of a fluorescent body and an antibody specifically binding to the antigen can be used as a labeling agent. Such a labeling agent can be produced by a known method, and a labeling agent for a specific capture target substance 85 is commercially available.

The SPR measurement method in the present embodiment may be the same as that for a labeling agent in a known SPR measurement method. A variety of known fluorescent body can be used in a case of being used for a fluorescence signal change. Typical fluorescent body include, for example, the following fluorescent substances. As the fluorescent substance, a rhodamine-based dye molecule, a squarylium-based dye molecule, a cyanine-based dye molecule, an aromatic ring-based dye molecule, an oxazine-based dye molecule, a carbopyronin-based dye molecule, a pyrromethene-based dye molecule, or the like can be used. Furthermore, as the fluorescent substance, Alexa Fluor® (manufactured by Invitrogen)-based dye molecules, BODIPY® (manufactured by Invitrogen)-based dye molecules, Cy® (manufactured by GE Healthcare)-based dye molecules, DY-based dye Molecules® (manufactured by DYOMICS), HiLyte® (manufactured by Anaspec)-based dye molecules, DyLight® (manufactured by Thermo Scientific)-based dye molecules, ATTO® (manufactured by ATTO-TEC)-based dye molecules, MFP® (manufactured by Mobitec)-based dye molecules or the like can be used. The general term of such a dye molecule is named based on the main structure (skeleton) in the compound or a registered trademark, and the range of the fluorescent substance belonging to each can be appropriately grasped by those skilled in the art without excessive trial and error. Furthermore, the fluorescent material is not limited to those described above. For example, rare earth complex-based fluorescent dyes such as Eu and Tb can also be the fluorescent dye used in the present embodiment. Rare earth complexes generally have a large difference between their excitation wavelengths (about 30 to 340 nm) and their emission wavelengths (near 65 nm for Eu complexes and near 545 nm for Tb complexes) and have a long fluorescence lifetime of several hundred microseconds or more. An example of a commercially available rare earth complex-based fluorescent dye includes ATBTA-Eu³⁺.

### (Coating and Adhesion of the Capture Substance to the Metal Thin Film)

The capture substance 81 can be applied to and immobilized to the metal thin film 82 as follows. For example, the same kind of capture substance 81 (for example, an antibody) is applied to each predetermined coating section (not shown) on the metal thin film 82 by an ink jet method, an applicator, a nozzle jet, direct dropping (a pipette, a quantitative weighing machine), or the like. After the application, washing is usually repeated to adhere the capture substance 81, but a method in which each capture substance 81 is solidified with one liquid is desirable.

### (Another Example of the Acquisition Unit: Transistor-Type Biosensor)

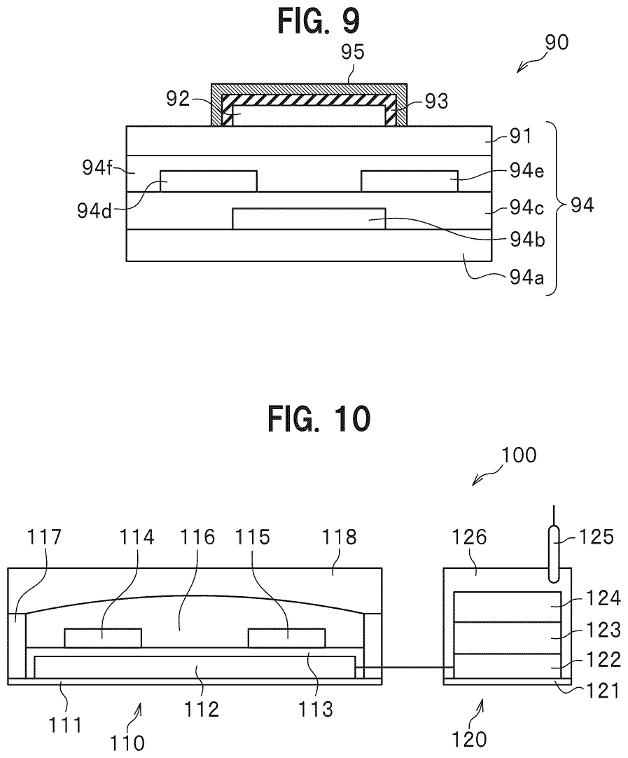

As described above, a detection method using a transistor-type biosensor can also be suitably applied as a high-sensitivity detection method in the present embodiment. FIG. 9 is a schematic view illustrating an example of a transistor-type biosensor.

As shown in FIG. 9, the transistor-type biosensor 90 has a structure in which a sensor part 93 described later is provided on a metal thin film 92 provided on an insulating film 91. A change in the threshold voltage, the drain current value, or the charge mobility, which is generated by a reaction occurring in the sensor part 93, is measured via the transistor part 94, and thus it is possible to detect the substance to be captured simply and with high sensitivity. The sensor part 93 may have the same configuration as the sensor part described in the SPR device 80. That is, the sensor part 93 can have a configuration in which the capture substance 95 is immobilized on its surface.

The transistor part 94 which can be used in the present embodiment can be formed of a known transistor structure. The transistor part 94 may be an inorganic transistor or may be an organic transistor. The transistor part 94 may have a top gate structure or a bottom contact structure. FIG. 9 illustrates a bottom contact structure as an example of the transistor part 94.

The transistor part 94 having a bottom-contact structure illustrated in FIG. 9 includes a gate electrode 94b formed on a substrate 94a. The transistor part 94 has a gate insulating film 94c formed so as to cover the gate electrode 94b on the substrate 94a. The transistor part 94 has a source electrode 94d and a drain electrode 94e separately formed on the gate insulating film 94c. The transistor part 94 has a semiconductor layer 94f formed so as to cover the source electrode 94d and the drain electrode 94e on the gate insulating film 94c. The transistor part 94 includes the insulating film 91 formed on the semiconductor layer 94f.

As the transistor part 94, an inorganic transistor can be suitably used from the viewpoint of durability. As the inorganic transistor, a commercially available transistor may be used.

Further, from the viewpoint of small size and simplicity, a thin-film transistor (TFT) is suitably used. In this case, as the substrate 94a, an inorganic material such as glass, ceramic, or metal can be applied. As the substrate 94a, an organic material such as a resin or paper can be used. The substrate 94a formed of these organic materials has flexibility.

In the case of an organic TFT, for example, resin such as polyethylene naphthalate, polyethylene terephthalate, polyethylene, polyimide, and polyparaxylylene (Parylene®), paper, or the like can be used as the substrate 94a.

Examples of the constituent material of the gate electrodes 94b include aluminum, silver, gold, copper, titanium, indium tin oxide (ITO), and PEDOT:PSS (abbreviation of a composite of poly (3,4-ethylene dioxythiophene) (PEDOT) and polystyrene sulfonate (PSS)) or the like can be used.

As the constituent material of the gate insulating film 94c, for example, silica, alumina, a self-assembled monolayer (SAM), polystyrene, polyvinyl phenol, polyvinyl alcohol, polymethyl methacrylate, polydimethylsiloxane, polysilsesquioxane, an ionic liquid, polytetrafluoroethylene (Teflon®AF, Cytop®), or the like can be used.

Examples of the constituent material of the source electrode 94d and the drain electrode 94e include gold, silver, copper, platinum, aluminum, and PEDOT: PSS or the like can be used.

In the case of an organic TFT, as the constituent material of the semiconductor layer 94f, as a P-type, pentacene, dinaphthothienothiophene, benzothienobenzothiophene (Cn-BTBT), TIPS pentacene (6, 13-bis [(triisopropylsilyl) ethynyl] pentacene), TES-ADT ([5, 11-bis (triethylsilyl ethynyl) anthradithiophene]), rubrene, P3HT (poly (3-hexylthiophene)), PBTTT (for example, poly [2,5-bis (3-hexadecylthiophen-2-yl) thieno [3,2-b]thiophene]), or the like can be used. In the case of an N-type, fullerene or the like can be used.

In addition, the method for producing the TFT may be a dry process such as a vapor deposition method or a sputtering method, may be coating by spin coating, bar coating, spray coating, or the like, or may be printing by various printing machines such as screen printing, gravure offset printing, letterpress reverse printing, and ink jet printing. According to the printing, it is possible to manufacture more efficiently at low cost.

### (Another Example of the Transistor-Type Biosensor)

Another example of the transistor-type biosensor will be described. FIG. 10 is a schematic view showing another example of the transistor-type biosensor.

As shown in FIG. 10, a transistor-type biosensor 100 according to another example is different from the transistor-type biosensor 90 (FIG. 9) in that the transistor-type biosensor 100 includes a transistor part 110 and a detection part 120, which are integrated with each other. The transistor-type biosensor 100 according to another example will be described focusing on differences from the transistor-type biosensor 90.

The transistor part 110 includes a gate electrode 112 formed on a substrate 111. The transistor part 110 includes a gate insulating film 113 formed so as to cover the gate electrode 112. The transistor part 110 includes a source electrode 114 and a drain electrode 115 that are separately formed on the gate insulating film 113. The transistor part 110 includes a semiconductor layer 116 formed so as to cover the source electrode 114 and the drain electrode 115 on the gate insulating film 113. The transistor part 110 has a lyophobic bank 117 covering the peripheries of the gate insulating film 113 and the semiconductor layer 116 on the substrate 111. The transistor part 110 includes a sealing film 118 formed on the semiconductor layer 116 and the lyophobic bank 117.

The detection part 120 includes a metal thin film 122 formed on the substrate 121. The detection part 120 has a sensor part 123 formed on the metal thin film 122. The detection part 120 includes a capture substance 124 formed on the sensor part 123. The detection part 120 includes a reference electrode 125 provided above the capture substance 124 so as not to be in direct contact with the capture substance 124. A droplet sample 126 is supplied between the capture substance 124 and the reference electrode 125.

The transistor part 110 and the detection part 120 are electrically connected by the gate electrode 112 of the transistor part 110 and the metal thin film 122 of the detection unit 120, and a change in signal detected by the detection part 120 can be acquired over time.

As a constituent material of the liquid repellent bank 117, for example, a fluororesin such as polytetrafluoroethylene, polyvinylidene fluoride, or polyvinyl fluoride can be used. The lyophobic bank 117 can be formed using an arbitrary dispenser device.

In addition, as a constituent material of the sealing film 118, for example, polytetrafluoroethylene, polyparaxylylene, or the like is exemplified.

The reference electrode 125 can be, for example, a silver/silver chloride electrode, a carbon electrode, a metal electrode deposited by physical vapor deposition (PVD, e.g., sputtering) or chemical vapor deposition (CVD), etc. In particular, a silver/silver chloride electrode or a carbon electrode is preferable because it can be formed by various printing methods and can be easily arranged arbitrarily. Further, the reference electrode 125 is preferably present in the same liquid as the liquid of the sensor part 123, and is preferably disposed in the vicinity of the sensor part 123 as shown in FIG. 10.

(Coating Metal Thin Film, Sensor Part, Capture Substance onto Metal Thin Film)

The metal thin films 92 and 122 in the transistor-type biosensors 90 and 100 can be similar to the metal thin film 82 of the SPR device 80.

The sensor units 93 and 123 in the transistor-type biosensors 90 and 100 can be similar to the sensor unit of the SPR device 80.

Coating the capture substances 95 and 124 onto the metal thin films 92 and 122 can also be performed in the same manner as in the SPR device 80.

### (Electrical Detection)

Electric signals from the transistor-type biosensors can be detected by sensors such as ISFET-F20 manufactured by ISFETCOM Co., Ltd. and Ion Image Sensor manufactured by Hamamatsu Photonics K.K., which are commercially available.

### (Analysis Unit)

Returning to FIG. 1, the analysis unit 3 will be described.

The analysis unit 3 analyzes at least one of the aggregation state or the surface state of the capture target substance from the signal change over time acquired by the acquisition unit 2. In addition, as described above, the particle size distribution can be observed by the analysis unit 3, and information on the surface state, crystallinity, and modifying chain can be acquired from the signal information. Further, the analysis means 3 can determine the features of the signal height, velocity, and signal behavior for each corresponding content, and acquire information in which each content is distinguished. The information obtained through analysis by the analysis unit 3 can be used by machine learning unit 4 of the learned model generating device 1A to be described later.

As the analysis unit 3, a general computer (general-purpose computer) can be used which includes input means such as a keyboard and a mouse (not illustrated), output means such as a monitor and a printer, storage means such as a hard disk drive (HDD), a solid state drive (SSD) and a read only memory (ROM) for storing programs and data, and a central processing unit (CPU) for executing programs and performing calculation processing. That is, by installing an arbitrary program for performing analysis in a storage unit of a general computer and executing the program, the computer can be used as the analysis unit 3.

### <Learned Model Generation Device>

Next, with reference to FIG. 11, a learned model generation device according to an embodiment of the present invention will be described. FIG. 11 is a schematic view illustrating a configuration of a learned model generation device 1A according to an embodiment of the present invention.

As shown in FIG. 11, the learned model generation device 1A includes an acquisition unit 2, an analysis unit 3, and a machine learning unit 4.

Note that since the acquisition unit 2 and the analysis unit 3 of the learned model generating apparatus 1A are the same as the acquisition unit 2 and the analysis unit 3, respectively, of the analysis system 1 described above, a description thereof will be omitted, and the machine learning unit 4 will be described.

### (Machine Learning Unit)

The machine learning unit 4 performs machine learning using a plurality of analysis results obtained by the analysis unit 3, and generates a learned model (prediction model). Similarly to the analysis unit 3 of the analysis system 1, the machine learning unit 4 can also use a general computer (general-purpose computer) comprising input means such as a keyboard and a mouse (not shown), output means such as a monitor and a printer, storage means such as an HDD, an SSD, and a ROM for storing programs, data, and the like, and a CPU for executing programs, calculation processing, and the like. That is, by installing any program for performing machine learning in a storage unit of a general computer and executing the program, the computer can be used as the machine learning unit 4.

### (Construction of a Prediction Model by Machine Learning)

From the features of the plurality of signals obtained from the acquisition unit 2, for example, for each aggregation size of the substance to be captured, a prediction model is constructed for the aggregation amount thereof. By combining the results of the plurality of prediction models, the distribution of the aggregation size and the aggregation amount of the capture target substance can be predicted. For example, a distribution diagram (image diagram) can be created in which the horizontal axis represents the aggregation size and the vertical axis represents the aggregation amount. These prediction models described above are constructed by performing, on test data in which the aggregation size and the aggregation amount of the substance to be captured are known in advance, machine learning in which the features of the plurality of signals obtained from the acquisition unit 2 are used as explanatory variables and the aggregation amount of the substance to be captured for each aggregation size is used as an objective variable.

The machine learning to be applied to the present embodiment may be supervised learning or unsupervised learning. The supervised learning is a learning method of learning a “relationship between an input and an output” from learning data with a correct answer label. The unsupervised learning refers to a learning method of learning the “structure of a data group” from learning data without a ground truth label.

Furthermore, the machine learning may be reinforcement learning, deep learning, or deep reinforcement learning. Note that the reinforcement learning is a learning method of learning an “optimal action sequence” by trial and error. Deep learning refers to a learning method in which, from a large amount of data, features included in the data are learned deeper (in deeper layers) in a stepwise manner. The deep reinforcement learning refers to a learning method in which the reinforcement learning and the deep learning are combined.

A general analysis method (algorithm) can be applied to the machine learning. For the machine learning, for example, a prediction model constructed by an analysis method selected from linear regression (multiple regression analysis, partial least squares (PLS) regression, LASSO regression, Ridge regression, principal component regression (PCR), and the like), random forest, a decision tree, a support vector machine (SVM), support vector regression (SVR); a (deep) neural network, and discriminant analysis can be applied.

As the explanatory variable, a numerical value representing a characteristic of each signal obtained from the acquisition unit 2 and a numerical value calculated from them can be used. Examples of the explanatory variable include the slope of the rise of each signal, the maximum value, the time until the signal rises, the dissociation constant (K_{d}), the association rate constant (kₒₙ), the dissociation rate constant (k_{off}), and the like.

It should be noted that the above-described embodiment is merely an example for embodying the present invention, and the technical scope of the present invention should not be interpreted in a limited manner by the above-described embodiment. The present proposal can be implemented in various forms without departing from the gist or the main features thereof. Although the aggregation amount for each aggregation size of the substance to be captured is used as an objective variable in the above embodiment, the objective variable is not limited thereto. The objective variable may be, for example, the surface structure of aggregation of the capture target substance, the mode of aggregation, the amount of charge, the magnitude of interaction, or the like. The explanatory variables are also not limited to the features of the signals and the values calculated therefrom. The explanatory variable may be, for example, the name of the capture substance of each sensor, the manufacturing time (manufacturing date) of the sensor, or the like.

### <Discrimination System>

Next, a discrimination system according to an embodiment of the present invention will be described with reference to FIG. 12. FIG. 12 is a schematic view illustrating a configuration of a discrimination system 1B according to an embodiment of the present invention.

As shown in FIG. 12, the discrimination system 1B uses the learned model generation device 1A described above and includes a discrimination unit 5. Note that since the learned model generating device 1A in the discrimination system 1B has already been described, a description thereof will be omitted, and the determination means 5 will be described.

Note that the analysis system 1 and the learned model generation device 1A described above are used by a provider who prepares a created learned model (program) and make users trying to discriminate unknown signal change over time use the learned model.

The discrimination unit 5 of the discrimination system 1B is used by users who want to discriminate an unknown signal change over time using a learned model prepared by a provider.

### (Discrimination Unit)

The discrimination unit 5 discriminates the acquired unknown signal change over time using the learned model generated by the learned model generation device 1A described above, and outputs a discrimination result of at least one of the aggregation state and surface state of the capture target substance having the unknown signal change over time. Note that the unknown signal change over time may be one acquired by the aforementioned acquisition unit 2, or one acquired by another acquisition means (another measurement device (SPR apparatus) or the like). The determination result can be, for example, a result of estimating and assuming what aggregation state and/or surface state the substance to be captured that is a determination target is in, or what aggregation state and/or surface state the substance to be captured will be in. The predicted result varies depending on the analysis method applied in machine learning, but can be obtained as a discrimination result of, for example, classification, regression, clustering, abnormality detection (outlier detection), or the like. For example, in a case where an unknown signal change over time (for example, a change in height, inclination, velocity, or the like) is input to the discrimination unit 5, an answer (predicted result (discrimination result)) of the aggregation distribution can be obtained using the learned model.

Similarly to the analysis unit 3 and the machine learning unit 4 of the analysis system 1, the discrimination unit 5 can use an input unit such as a keyboard and a mouse (not illustrated), an output unit such as a monitor and a printer, a storage unit such as an HDD, an SSD, and a ROM that store a program, data, and the like, and a general computer (general-purpose computer) comprising a CPU that executes a program, performs calculation processing, and the like. That is, by installing any program for performing the discrimination unit 5 in a storage unit of a general computer and executing the program, the computer can be used as the discrimination unit 5.

### <Analysis Method, Learned Model Generation Method, and Discrimination Method>

Next, an analysis method, a learned model generation method, and a discrimination method according to an embodiment of the present invention will be described with reference to the drawings as appropriate.

### (Analysis Method)

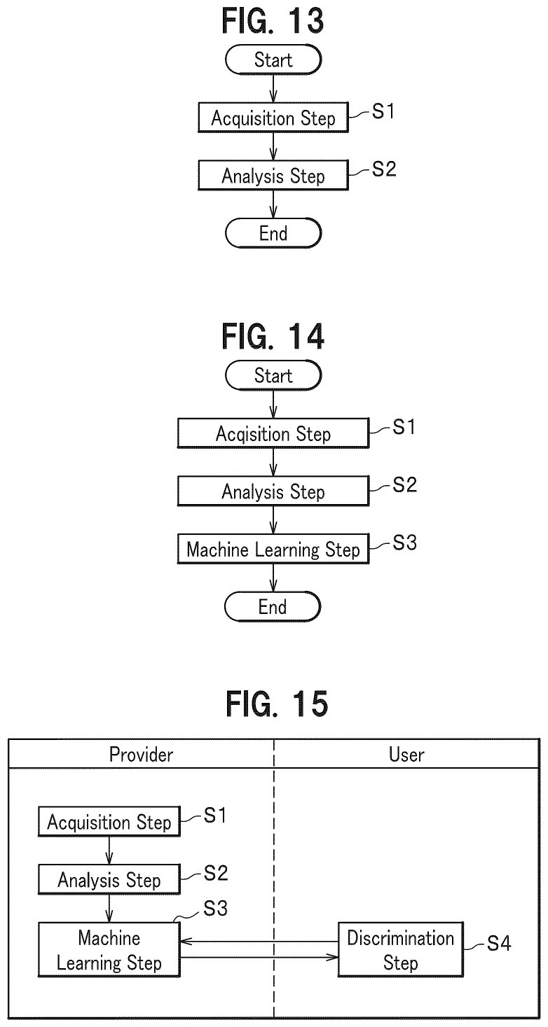

FIG. 13 is a flowchart illustrating an analysis method according to an embodiment of the present invention. As shown in FIG. 13, the analysis method includes an acquisition step S1 and an analysis step S2.

The acquisition step S1 is a step of acquiring signal changes over time due to interactions between the capture target substances and the capture substances by an electrical or optical detection method.

The analysis step S2 is a step of analyzing at least one of the aggregation state and the surface state of the capture target substance from the time-dependent signal change acquired in the acquisition step S1.

Note that the acquisition step S1 can be performed by the acquisition unit 2 described above, and the analysis step S2 can be performed by the analysis unit 3 described above.

### (Learned Model Generation Method)

FIG. 14 is a flowchart illustrating a learned model generation method according to an embodiment of the present invention.

As illustrated in FIG. 14, the learned model generation method includes an acquisition step S1, an analysis step S2, and a machine learning step S3. Since the acquisition step S1 and the analysis step S2 of the learned model generation method are the same as the acquisition step S1 and the analysis step S2 of the above-described analysis method, description thereof will be omitted, and the machine learning step S3 will be described.

The machine learning step S3 is a process of performing machine learning using a plurality of analysis results obtained by the analysis step S2 and generating a learned model.

Note that the machine learning step S3 can be performed by the aforementioned machine learning unit 4.

### (Discrimination Method)

FIG. 15 is a flowchart illustrating a discrimination method according to an embodiment of the present invention. The discrimination method according to the present embodiment includes a discrimination step S4 of discriminating the acquired unknown signal change over time using the learned model generated by the learned model generation method described with reference to FIG. 14 and outputting the discrimination result of at least one of the aggregation state or the surface state of the capture target material having the unknown signal change over time. In the discrimination step S4, a user who wants to obtain the discrimination result inputs an unknown signal change over time to the learned model to obtain the discrimination result. Note that the acquisition step S1, the analysis step S2, and the machine learning step S3 in the discrimination method are performed by a provider who allows the created learned model (program) to be used by the user. As shown in FIG. 15, the discrimination methods according to the present embodiment are performed in the order of an acquisition step S1, an analysis step S2, and a machine-learning step S3 as a whole, and these steps are performed before the discrimination step S4 is performed.

Since the acquisition step S1, the analysis step S2, and the machine learning step S3 of the discrimination method are the same as the acquisition step S1, the analysis step S2, and the machine learning step S3 of the learned model generation method, respectively, the description thereof will be omitted.

The discrimination step S4 can be performed by the discrimination unit 5 described above.

The analysis system, the learned model generating apparatus, the determination system, the analysis method, the learned model generating method, and the determination method according to the present embodiment described above include the acquisition means and the acquisition process and the analysis means and the analysis process described above. Therefore, in any of these methods, a signal change over time due to the interaction between the capture target substance and the capture substance is obtained by an electrical or optical detection method, and at least one of the aggregation state and the surface state of the capture target substance can be analyzed from the signal change over time. Therefore, both of them can quantify at least one of the aggregation state and the surface state with high sensitivity.

### Example 1

### Detection by Electricity: Aβ1-42

### (Production of Aβ1-42 Aggregate)

The present inventors dissolved a dry material of Aβ1-42 (totally synthetic peptide; manufactured by Peptide Institute, Inc) in dimethylsulfoxide (DMSO) so as to be a 1 mM, and diluted with a phosphate buffer to prepare a low aggregation sample.

Furthermore, the present inventors treated Aβ1-42 dissolved in DMSO with ultrasonic waves for 10 minutes, diluted the solution with a phosphate buffer solution, and then left at rest at 37° C. for 24 hours, and defined the sample as a highly aggregated sample.

### (Production of Extension Gate)

The present inventors pattern-deposited Au on the surface of a polyethylene naphthalate (PEN) film as a substrate using a deposition apparatus (manufactured by ALS Technology Co. Ltd). The thickness of the deposited Au film was 100 nm. A reaction part and an extension part were prepared as an Au pattern, and the reaction part had a 6 mm×6 mm square shape in order to prepare a reaction field described below.

The present inventors immersed the reaction part of the Au film in a hexane solution containing a concentration of 1 mM 10-carboxy-1-decanethiol (Dojindo Laboratories) at 37° C. for 2 hours to form a SAM film. Thereafter, the SAM film was washed with ethanol and ultrapure water.

The present inventors dropped 30 μl of 100 mM MES buffer (Dojindo Laboratories) containing 5 mM Sulfo-NHS (N-hydroxysulfosuccinimide, manufactured by Thermo Scientific), 40 mM N,N′-diisopropylcarbodiimide (manufactured by Tokyo Chemical Industry Co., Ltd.), and 0.5 M NaCl (manufactured by Kanto Chemical Co., Inc) onto the reaction part on which the SAM film had been formed and left at rest at 37° C. for 15 minutes.

After the reaction, the solution added dropwise was removed by the present inventors, and then 50 μl of a carbonate buffer (15 mM Na₂CO₃ (manufactured by Kanto Chemical Co., Inc), 35 mM NaHCO₃ (manufactured by Kanto Chemical Co., Inc)) containing streptoavidin (manufactured by Nacalai Tesque, Inc) at a concentration of 0.5 mg/ml was added dropwise thereto and left at rest at 37° C. for 2 hours.

The present inventors removed the dropped liquid, then added dropwise 20 μl of D-PBS buffer (manufactured by FUJIFILM Wako Pure Chemical Industries, Ltd) containing 2-aminoethanol at a concentration of 1M, and left the mixture to rest at 37° C. for 15 minutes.

20 μl of D-PBS buffer containing Tween20 (manufactured by Kanto Chemical Co., Inc) at a concentration of 0.05 wt % and BSA (manufactured by FUJIFILM Wako Pure Chemical Corporation) at a concentration of 0.1 wt % was added dropwise to the plate, and the plate was left at rest at 37° C. for 15 minutes.

After removing the dropped liquid, washing with ultrapure water was performed, and 20 μl of a 3-fold dilution of the biotin-conjugated antibodies (BAN50) contained in a high-molecular amyloid β oligomer ELISA kit Wako Ver. 2 (manufactured by FUJIFILM Wako Pure Chemical Corporation) was added dropwise thereto, and the plate was left at rest at 37° C. for 30 minutes.

The present inventors removed the dropped liquid, and then washed with D-PBS buffer containing BSA at a concentration of 0.1 wt % and dimethylsulfoxide (DMSO) at a concentration of 5 wt %.

### (Titration)

The present inventors prepared 700 μl of D-PBS buffer containing BSA at a concentration of 0.1 wt % and dimethylsulfoxide (DMSO) at a concentration of 5 wt % in a microtube, and set a SAM film of an extension gate and a reference electrode Ag/AgCl (RE-1, manufactured by BAS Inc).

As illustrated in FIG. 6 described above and referenced to, the present inventors connected a FET (manufactured by 2SK241 Toshiba Corporation) and a semiconductor parameter analyzer (manufactured by Keysight Technologies).

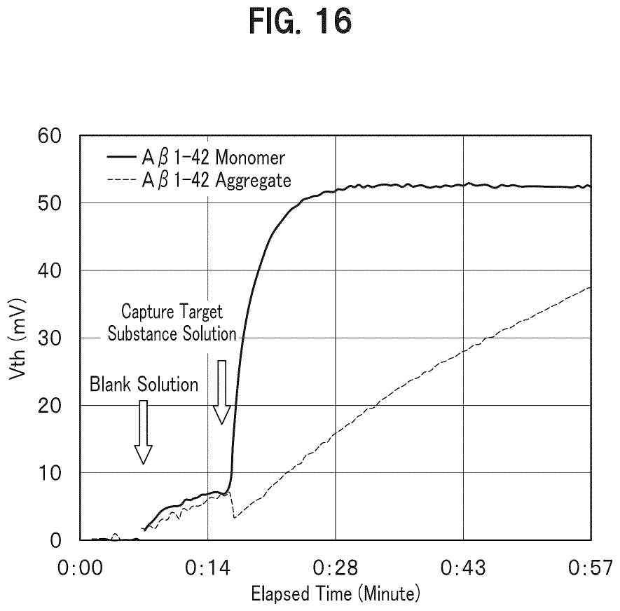

For stabilization, the present inventors added dropwise a blank solution (initial buffer (D-PBS buffer)) for 2 hours after setting (FIG. 16 illustrates a state where measurement is started (0:00) a little before 2 hours from the start of the dropwise addition of the blank solution and the blank solution is added dropwise until about 0:07).

Next, after 2 hours from setting, the present inventors added dropwise 100 μl of a low aggregation sample (capture target substance solution) having a concentration of 50 μM diluted with D-PBS (the sample was added dropwise at a timing of about 0:15 in FIG. 16).

Next, the inventors measured a change in the drain-source current Ids while fixing the drain-source voltage Vds at 1 V and changing the gate-source voltage Vgs between −1.5 V and 1.5 V, and measured a change in the threshold value voltage Vth over time every 30 seconds for about 40 minutes (until 0:57 in FIG. 16). That is, the threshold value voltage Vth was intermittently measured every second.

Further, the present inventors also performed a titration operation on a highly aggregated sample (concentration 50 μM) in the same manner as described above. FIG. 16 indicates the results. FIG. 16 is a graph showing temporal changes in threshold value voltage Vth of the low aggregation sample (Aβ1-42 monomer) and the high aggregation sample (Aβ1-42 aggregate).

### (Result)

As shown in FIG. 16, it was confirmed and analyzed that there is a difference in signal change over time measured by detection by the electricity of the FET between the low aggregation sample (Aβ1-42 monomer) and the high aggregation sample (Aβ1-42 aggregate). Since such a difference has been confirmed and analyzed by the FET, it is also possible to acquire and analyze different signal changes over time between a low aggregation sample (Aβ1-42 monomer) and a high aggregation sample (Aβ1-42 aggregate) by optical detection, for example, by an SPR apparatus.

### Example 2

### Detection by Electricity: Nanomaterial

### (Production of Extension Gate)

The present inventors pattern-deposited Au on a surface of a PEN film serving as a base material, using a vapor deposition apparatus (manufactured by ALS Technology, Co., Ltd.). The thickness of the deposited Au film was 100 nm. A reaction part and an extension part were prepared as an Au pattern, and the reaction part had a 6 mm×6 mm square shape in order to prepare a reaction field described below.

The present inventors immersed the reaction part of the Au film in an ethanol solution containing a concentration of 0.1 mM Biotin-SAM Formation Reagent (Dojindo Laboratories) at 37° C. for 2 hours to form a biotin-bonded SAM film. Thereafter, the present inventors washed with D-PBS buffer.

### (Titration)

The present inventors prepared 700 μl of a D-PBS buffer containing 0.1 wt % BSA at a concentration of 0.1 wt % and glycerol at a concentration of 2 wt % in a microtube, and set a SAM film of an extension gate and a reference electrode Ag/AgCl (RE-1, manufactured by BAS Inc).

Then, the present inventors connected an FET (manufactured by Toshiba Corporation, 2SK241) and a semiconductor element parameter analyzer (manufactured by Keysight Technology) as shown in FIG. 6 described above and referenced to.

For stabilization, the present inventors added dropwise a blank solution (initial buffer (D-PBS buffer)) for 2 hours after setting.

Next, two hours after the setting, the present inventors added dropwise 70 μl of Streptavidin, Gold Colloidal Particle 5 nm (OD=3, manufactured by Cosmo Bio, Co., Ltd.) (target solution).

Next, the present inventors measured a change in the drain-source current Ids while fixing the drain-source voltage Vds at 1 V and changing the gate-source voltage Vgs between −1.5 V and 1.5 V, and measured a change in the threshold voltage Vth over time every 30 seconds for about 40 minutes.

Further, the present inventors performed the same titration operation for Streptavidin, Gold Colloidal Particle 15 nm and 60 nm (OD=3, manufactured by Cosmo Bio Co., Ltd).

### (Result)

It was confirmed that a signal change over time corresponding to the particle diameter could be obtained also for the nanomaterial by detection using electricity of the FET, and it was confirmed and analyzed that there was a difference in these signal changes. In addition, the concentration of the particles was adjusted from the ODs of 5 nm, 15 nm, 60 nm Streptavidin, and Gold Colloidal Particle, the same examination was performed, and a difference was detected in each signal change.

### Example 3

### Machine Learning Model and Classification Prediction: Machine Learning Model and Classification Prediction: Aβ1-42

In Example 3, a low aggregation sample (Aβ1-42 monomer) and a high aggregation sample (Aβ1-42 aggregate) were prepared in the same manner as in Example 1 except that various concentrations were used, and titration was performed to obtain a signal change over time measured by electrical detection of the FET. FIG. 17 indicates the results. FIG. 17 is a graph showing changes over time in the threshold value voltage Vth of a low-aggregation sample (Aβ1-42 monomer) and a high-aggregation sample (A β1-42 aggregate) at different concentrations. Note that FIG. 17 shows the measurement results after the capturing target substance liquid was dropped.

As shown in FIG. 17, also in Example 3, it could be confirmed that there was a difference in signal change over time measured by electrical detection of the FET between the low aggregation sample (A β1-42 monomer) and the high aggregation sample (A β1-42 aggregate).

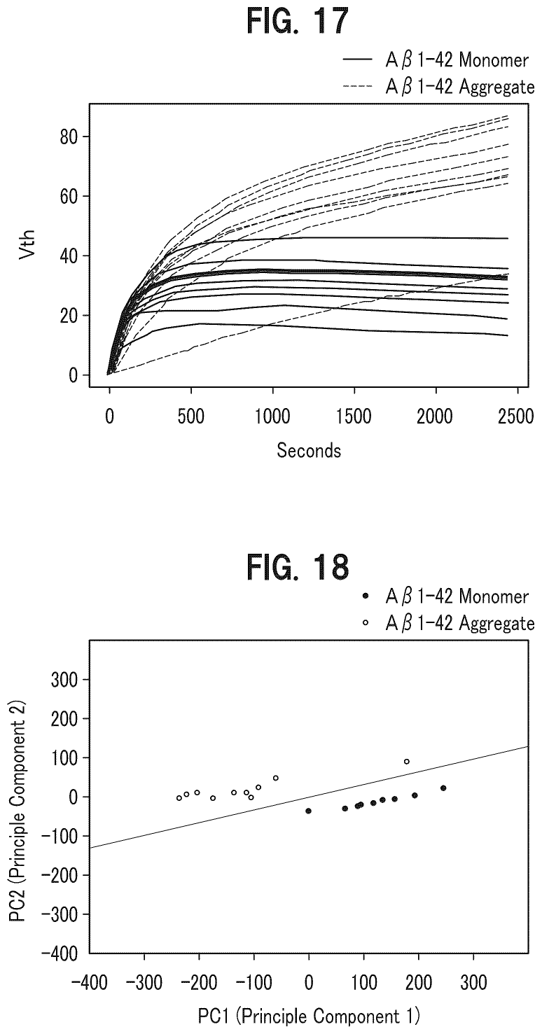

Next, using these measurement results in Example 3, the present inventors performed learning and classification prediction of a support vector machine (SVM) that is a machine learning model, with data obtained by dimensionally compressing the data on the change over time in threshold value voltage Vth by principal component analysis as an explanatory variable, and with whether it was a low aggregation sample or a high aggregation sample as an objective variable. FIG. 18 indicates the results. FIG. 18 is a graph showing the results of learning and classification prediction of an SVM, which is a machine learning model, using explanatory variables obtained by dimensionally compressing the data on the change over time in the threshold voltage Vth by principal component analysis, and objective variables indicating whether the sample is a low-aggregation sample or a high-aggregation sample. As shown in FIG. 18, a machine learning model (learned model) was generated, and a low aggregation sample and a high aggregation sample could be classified (discriminated) using the machine learning model.

Also in Example 3, since such a difference was confirmed and analyzed by the FET, it is possible to acquire and analyze different signal changes over time between the low aggregation sample (Aβ1-42 monomer) and the high aggregation sample (Aβ1-42 aggregate) also by optical detection, for example, by the SPR apparatus. Furthermore, as explanatory variables in machine learning, in addition to the threshold value voltage Vth, for example, a kₒₙ, k_{off}, a drain-source voltage Vds, a numerical value or a character string representing the amount or a property of a used reagent, a numerical value or a character string representing a property of an FET, or numerical values obtained by processing those can be used. Furthermore, as an objective variable in machine learning, not only a qualitative variable such as whether a sample is a low aggregation sample or a high aggregation sample, but also a quantitative variable such as the amount or ratio of a sample in each aggregation state can be used. As a model for machine learning, not only SVM but also classification models such as logistic regression and discrimination analysis can be used, for example. Furthermore, as a model for machine learning, a prediction model such as linear regression, partial least squares regression (PLS regression), or least absolute shrinkage and selection operator regression (Lasso regression) can be used. Furthermore, as a model for machine learning, a decision tree, a random forest, a deep neural network, or the like can be used.

### [Discussion]

From the results of Example 1, Example 2, and Example 3, it was confirmed that even for a minute and low-concentration object (capture material) such as an Aβ protein fragment (Aβ1-42), streptavidin, Gold Colloidal Particle 5 nm, 15 nm, or 60 nm, a signal change over time can be acquired and analyzed by an electrical or optical detection method. Then, it was confirmed that the signal change over time differed depending on the state of the object, for example, the state of a monomer, an aggregate, or the like.

Then, it was confirmed that a learned model can be created by collecting a large number of data of signal changes over time, which are different depending on the object and its state, and performing machine learning with a computer or the like (as described above, for example, learning and classification prediction of SVM can be performed). Further, it was confirmed that analysis (discrimination) such as classification, regression, clustering, abnormality detection (outlier detection) can be performed on the acquired unknown continuous signal change (data) using the created learned model.

### REFERENCE SIGNS LIST

1 Analysis system
1A Learned model generation device
1B Discrimination system
2 Acquisition unit
3 Analysis unit
4 Machine learning unit
5 Discrimination unit
S1 Acquisition step
S2 Analysis step
S3 Machine learning step
S4 Discrimination step

What is claimed is:

1. An analysis system comprising
an acquisition unit configured to acquire a signal change over time due to an interaction between a capture target substance and a capture substance by an electrical detection method or an optical detection method, and
an analysis unit configured to analyze at least one of an aggregation state and a surface state of the capture target substance from the signal change over time acquired by the acquisition unit.

2. A learned model generation device comprising
an acquisition unit configured to acquire a signal change over time due to an interaction between a capture target substance and a capture substance by an electrical detection method or an optical detection method,
an analysis unit configured to analyze at least one of an aggregation state and a surface state of the capture target substance from the signal change over time acquired by the acquisition unit, and
a machine learning unit configured to perform machine learning by using a plurality of analysis results obtained by the analysis unit to generate a learned model.

3. A discrimination system comprising a discrimination unit configured to
discriminate an acquired unknown signal change over time by using the learned model generated by the learned model generation device according to claim 2, and
output a discrimination result of at least one of an aggregation state and a surface state of a capture target substance having the unknown signal change over time.

4. An analysis method comprising
an acquisition step of acquiring a signal change over time due to an interaction between a capture target substance and a capture substance by an electrical detection method or an optical detection method, and
an analysis step of analyzing at least one of an aggregation state and a surface state of the capture target substance from the signal change over time acquired in the acquisition step.

5. A learned model generation method comprising
an acquisition step of acquiring a signal change over time due to an interaction between a capture target substance and a capture substance by an electrical or optical detection method,
an analysis step of analyzing at least one of an aggregation state and a surface state of the capture target substance from the signal change over time acquired in the acquisition step, and
a machine learning step of performing machine learning by using a plurality of analysis results obtained in the analysis step to generate a learned model.

6. A discrimination method comprising a discrimination step of
discriminating an acquired unknown signal change over time using the learned model generated by the learned model generation method according to claim 5, and
outputting a discrimination result of at least one of an aggregation state and a surface state of a capture target substance having the unknown signal change over time.
